(19) United States
(12) Patent Application Publication
(10) Pub. No.: US 2009/0228021 A1
(43) Pub. Date: Sep. 10, 2009

(54) MATRIX MATERIAL

(76) Inventor: Jeffrey C. Leung, Raleigh, NC (US)

Correspondence Address: ANGIOTECH, P. O. BOX 2840, NORTH BEND, WA, 98045, US

(21) Appl. No.: 12/043,391

(22) Filed: Mar. 6, 2008

(51) Int. Cl. A61B 17/04; A61B 17/06

(52) U.S. Cl. 606/139; 606/151

(57) ABSTRACT

A matrix material is provided for implanting in tissue in surgical applications. The matrix material includes a body portion and at least one suture attached to the body portion so that the suture extends outwardly from the body portion. The suture includes an elongated suture body having a first distal end and a second proximal end, and a plurality of barbs projecting from the elongated suture body. Each barb faces in a direction and is adapted for resisting movement, when in tissue, in an opposite direction from the direction in which the barb faces. Also provided is a surgical method for suturing to tissue a matrix material. Also, the matrix material may be packaged inside sterile packaging.

### BACKGROUND

This invention relates generally to a matrix material that typically is surgically placed within a living body, such as an animal or a human, and more particularly, to a matrix material for use in surgical procedures and in various laboratory procedures. In one embodiment, the matrix material may be a hernia repair material, such as that useful in hernia repair surgery. In another embodiment, the matrix material may be biological scaffolding, such as that useful for a surgical implant or for growing tissue in an environment outside the patient's body, for instance, in a laboratory.

Traditionally, hernia repair had been accomplished using a suture by approximating the transversus abdominis tendon to the iliopubic tract, inguinal ligament, or Cooper's ligament. However, these structures are not normally in apposition and thus their approximation is associated with tension on the suture line. Also, inguinal hernias can be caused by a metabolic disorder involving collagen turnover of the transversalis fascia, which weakens the transversalis fascia and thus use of such defective tissue in hernia repair would be counterproductive.

Accordingly, surgically implantable matrix materials have been developed, such as mesh or cloth (i.e., surgical mesh, mesh graft, and the like). Matrix materials typically are intended for permanent placement within a patient's body space, for instance for the repair of inguinal and other abdominal wall hernias or other types of hernias as known in the art. As the various materials are intended to provide permanent repair, nonabsorbable (also called non-biodegradable) materials are generally used. Absorbable (also called biodegradable, or called bioabsorbable) matrix materials are also used as appropriate.

Prior to surgery, spikes (as shown in U.S. Pat. No. 5,766,246, issued Jun. 16, 1998 to Mulhauser et al.) or conventional standard sutures (as shown in Medical and Health Care, an advertisement by Gore Creative Technologies for their GORE® surgical hernia barrier fabric on their www.gore.com web site) are placed by the surgeon on the hernia repair matrix material for use in approximating the material to tissue, thus keeping the material in place, after the surgeon has positioned the material at the site of the hernia defect.

Matrix material useful as biological scaffolding is well known in the medical arts for use in implants. Such scaffolding material is intended for insertion into the patient's body to provide a biological or chemical agent, such as a cancer treating agent, to the patient's body, or to grow tissue or another biological material inside the patient's body. Scaffolding material is also intended for growing tissue, for instance tissue cells or other biological materials, in a laboratory environment, and then later on, the resultant tissue cells or biological material is inserted into the patient's body.

Like hernia repair material, scaffolding material can be nonabsorbable or absorbable. Absorbable scaffolds for tissue engineering are described in U.S. Pat. No. 6,696,575, issued Feb. 24, 2004, to Schmidt et al. This patent describes a polymer having pyrrole units, thiophene units, and ester units, the polymer being a biodegradable electrically conducting polymer useful for scaffolding.

Composite scaffolds for tissue engineering are described in U.S. Pat. No. 6,692,761, issued Feb. 17, 2004, to Mahmood et al. This patent describes a scaffold having a two-layer system of a biodegradable layer of a copolymer of a polyalkylene glycol and an aromatic polyester, and a non-biodegradable layer of a ceramic material.

Of interest, U.S. Pat. No. 6,689,166, issued Feb. 10, 2004, to Laurencin et al., describes a tissue engineering scaffold of biocompatible nonwoven nanofibrils that are a non-biodegradable polymer chosen from polyethylenes and polyurethanes or a bio-degradable polymer chosen from poly(lactic acid-glycolic acid), poly(lactic acid), poly(glycolic acid), poly(glaxanone), poly(orthoesters), poly(pyrrolic acid), and poly(phosphazenes).

As further background, it is interesting to note that U.S. Pat. No. 6,251,143, issued Jun. 26, 2001, to Schwartz et al., describes a device for placement between damaged cartilage and healthy bone. The device includes an insert that is a chondrogenic growth-supporting matrix of bioabsorbable material. The insert can contain various repair factors within the insert's matrix, such as fibroblast growth factors, transforming growth factor-beta, insulin, insulin-like growth factor 1 & 2 (IGF), platelet-derived growth factor, and other growth factors well known in the medical arts.

Also interesting as background is U.S. Pat. No. 6,716,246, issued Apr. 6, 2004, to Gonzalez. This patent describes a device including a scaffold that is a porous body having a cavity for housing a plunger. The porous body is a cylindrical grid made, for instance, of stainless steel. The device is implanted in a patient, and the patient's body by natural action makes the patient's own fibrocollagen form and overlay the porous body. An incision is made in the patient, and the plunger is removed, leaving a fribrocollagenous tube which is then implanted with a biological factor for producing cells.

Additional background on scaffolds is in U.S. Pat. No. 6,548,002, issued Apr. 15, 2003, to Gresser et al. and U.S. Pat. No. 6,582,960, issued Jun. 24, 2003, to Martin et al. The former patent discloses a method for making a resorbable interbody fusion device for use in spinal fixation, where the device is made from a material comprising 25-100% of a polymer that degrades by hydrolysis to produce acidic products. The latter patent discloses a method for expanding chondrocytes in the presence of fibroblast growth factor 2 on a cartilage scaffolding matrix, for use in regeneration of cartilage tissue.

The disclosures of all patents and published patent applications mentioned herein are incorporated by reference in their entirety.

Conventional methods for approximating matrix material to tissue using a standard suture are difficult and inefficient because the procedure requires manipulation of the suture to attach to the matrix and secure the tissue in place with knots. The procedure is time-consuming, particularly in microsurgery, laparoscopic surgery, endoscopic surgery, and arthroscopic surgery where there is insufficient space for proper manipulation of the suture. Moreover, suture knots, from use of standard sutures, create localized pressure points or sites, which deform the matrix material and can alter cellular activities in a scaffold. Thus, wound repair or healing can be significantly compromised. For these reasons, delicate scaffolds containing tissue cells are frequently implanted without any suturing despite the risk of inadequate fixation to the body.

For the foregoing reasons, there is a need for an improved matrix material for use in surgical procedures. The new matrix material should eliminate the need for tying knots in the suture to hold the matrix material in tissue. The new matrix material in surgical applications should allow a surgeon to approximate tissue to matrix material in an efficient manner. A particularly useful new matrix material would be used in surgical applications where space is limited such as microsurgery, laparoscopic surgery, endoscopic surgery, and arthroscopic surgery.

### SUMMARY

According to the present invention, a matrix material is provided for implanting in tissue in surgical applications. The matrix material comprises a body portion and at least one suture attached to the body portion so that the suture extends outwardly from the body portion. The suture comprises an elongated suture body having a first distal end and a second proximal end, and a plurality of barbs projecting from the elongated suture body. Each barb faces in a direction and is adapted for resisting movement, when in tissue, in an opposite direction from the direction in which the barb faces. The matrix material may comprise a hernia repair material, such as that useful in hernia repair surgery for a patient, or may comprise a scaffolding material, such as that useful for a surgical implant or for growing tissue in an environment outside the patient's body, for instance, a laboratory.

Also, the present invention provides a method for suturing to tissue a matrix material, the suturing method comprising the steps of: (a) providing a matrix material including a body portion and at least one suture attached to the body portion so that the suture extends outwardly from the body portion, the at least one suture comprising an elongated suture body having a first distal end and a second proximal end and a plurality of barbs projecting from the elongated suture body, each barb facing in a direction and being adapted for resisting movement, when in tissue, in an opposite direction from the direction in which the barb faces; (b) positioning the body portion in the tissue; (c) inserting the distal end of the at least one suture into the tissue; (d) pushing the distal end of the at least one suture through the tissue until the distal end of the suture extends out of the tissue at an exit point spaced from the point of insertion into the tissue; and (e) gripping the distal end of the at least one suture and pulling the end out of the tissue for drawing the suture through the tissue while approximating the body portion adjacent a selected position in the tissue and leaving a length of the at least one suture in the tissue.

Further according to the present invention, a package is provided, the package comprising a container having a sterile inside environment and a matrix material disposed inside the container. The matrix material comprises a body portion and at least one suture attached to the body portion so that the suture extends outwardly from the body portion. The suture comprises an elongated suture body having a first distal end and a second proximal end, and a plurality of barbs projecting from the elongated suture body. Each barb faces in a direction and is adapted for resisting movement, when in tissue, in an opposite direction from the direction in which the barb faces.

### BRIEF DESCRIPTION OF DRAWINGS

For a more complete understanding of the present invention, reference should now be had to the embodiments shown in the accompanying drawings and described below. In the drawings:

FIG. 1 is a perspective view of an embodiment of a matrix material according to the present invention;

FIG. 2 is a perspective view of another embodiment of a matrix material according to the present invention;

FIG. 3 is a perspective view of an embodiment of a matrix material according to the present invention for use a biological scaffolding;

FIG. 4 is a perspective view of another embodiment of a matrix material according to the present invention for use as biological scaffolding;

FIG. 5 is a schematic view of a package comprising a container having a sterile inside environment, and having the matrix material as shown in FIG. 1 disposed inside the container; and

FIG. 6 is a schematic view of a package comprising a container having a sterile inside environment, and having the matrix material as shown in FIG. 2 disposed inside the container.

### DESCRIPTION

As used here with respect to the present invention, the term “tissue” includes, but is not limited to, soft tissue and/or fibrous tissue, such as skin, fat, fascia, bone, nerves, blood vessels, tendons, ligaments, cartilage, muscle, skin, organs, or combinations thereof.

The term “matrix material” as used here with respect to the present invention generally includes, but is not limited to, any material that is surgically implantable and intended for temporary or permanent placement within a patient's body space, such as surgical mesh, mesh graft, sheeting, cloth, hernia repair material, scaffolding material (for instance, material used for biological scaffolding), and the like, or combinations thereof.

The term “polymer” as used here with respect to the present invention generally includes, but is not limited to, homopolymers, copolymers (such as block, graft, random and alternating copolymers), terpolymers, et cetera, and blends and modifications thereof. Furthermore, the term “polymer” shall include all possible structures of the material. These structures include, but are not limit to, isotactic, syndiotactic, and random symmetries.

The term “laboratory” as used here with respect to the present invention generally includes, but is not limited to, any environment outside the patient's body, for instance, a hospital laboratory.

The term “diameter” as used here with respect to a suture of the present invention is intended to mean the transverse length of the cross section of the suture, regardless of whether the cross section is circular or some other shape, for instance, marquise, oval, triangular, cruciform, square, rectangular, parallelepiped, trapezoidal, rhomboidal, pentagonal, hexagonal, heptagonal, octagonal, etc.

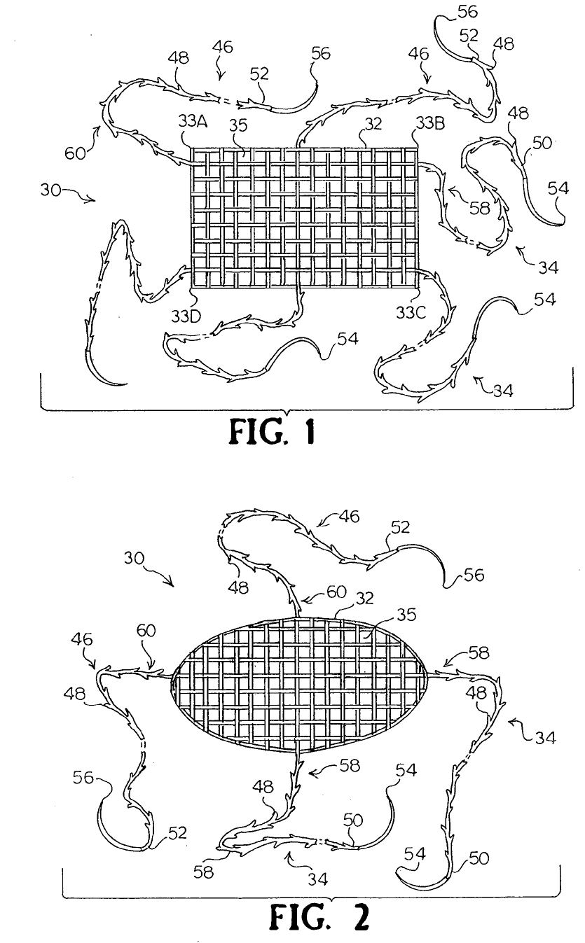

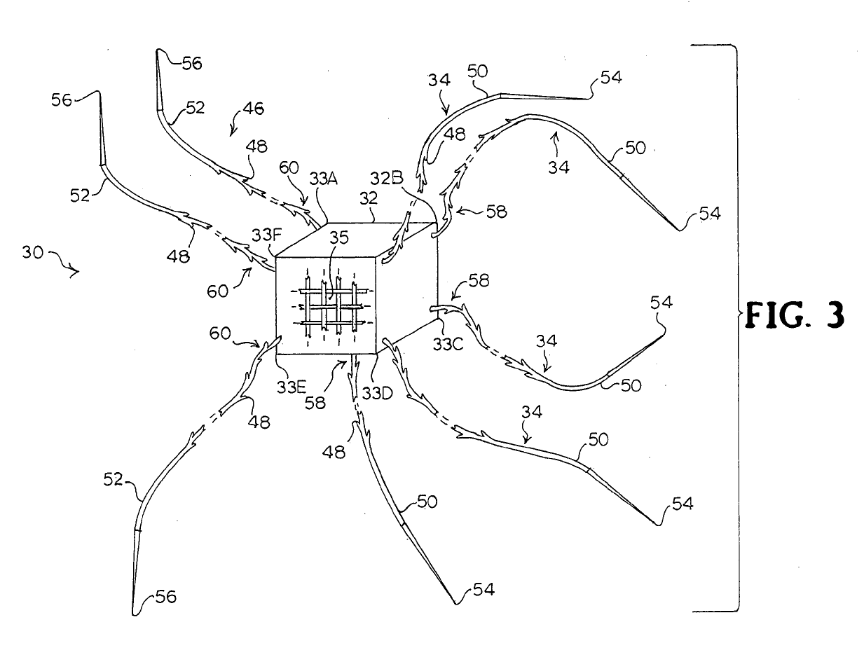

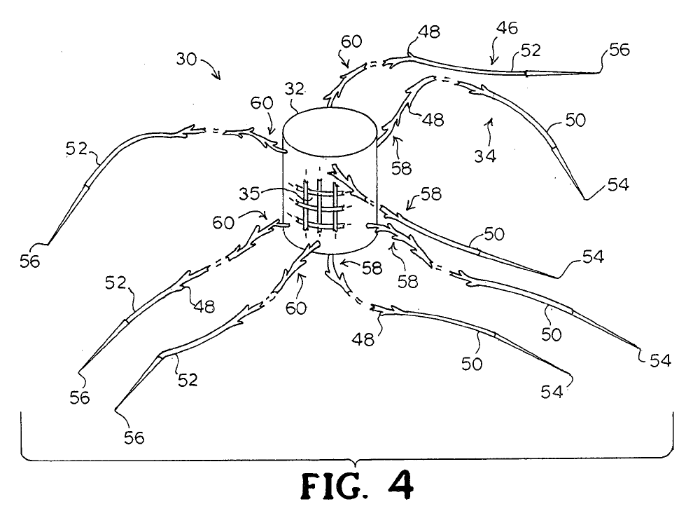

Referring now to the drawings, wherein like reference numerals designate corresponding or similar elements throughout the several views, there is shown in FIGS. 1-4 a matrix material according to the present invention and generally designated at 30. The matrix material 30 includes a body portion 32 and a suture portion 34. The body portion 32 comprises a matrix material as defined herein. The body portion 32 is shown as generally rectangular in FIG. 1, generally oval in FIG. 2, generally cubic in FIG. 3, and generally cylindrical in FIG. 4. It is understood, however, that the body portion 32 may be any shape, including, but not limited to, generally marquise, round, oval, triangular, cruciform, square, rectangular, parallelogram, trapezoidal, rhomboidal, pentagonal, hexagonal, heptagonal, octagonal, spherical, ellipsoidal, conical, cubic, cylindrical, etc.

The suture portion 34 of the matrix material 30 comprises at least one suture. Further, at least one suture of suture portion 34 comprises an elongated body 46 and a plurality of barbs 48 projecting from the body 46. In the embodiment shown in the FIGs., the suture body 46 is circular in cross section. It is understood, however, that the suture could also have a non-circular cross section shape that could increase the surface area and facilitate the formation of the barbs. Other cross sectional shapes may include, but are not limited to, marquise, oval, triangular, square, rectangular, parallelepiped, trapezoidal, rhomboidal, pentagonal, hexagonal, heptagonal, octagonal, cruciform, etc. The diameter of the suture body 46 may range from about 0.001 mm to about 5.0 mm, more particularly about 0.005 mm to about 1 mm. The typical diameter ranges from about 0.01 mm to about 0.5 mm. The length of each suture of the suture portion 34 can vary depending on several factors, including the desired surgical procedure or scaffolding application, the type of tissue to be sutured, the location of the surgical site, and the like. An appropriate length is selected for achieving suitable results in a particular application. Typical lengths range from about 1 cm to about 30 cm, more particularly from about 2 cm to about 22 cm.

The plurality of barbs 48 is disposed in an axially spaced arrangement along the length of the suture body 46. The barbs 48 are yieldable in one direction toward the body 46 and are generally rigid in an opposite direction to prevent the suture body 46 from moving in the tissue in the opposite direction. More particularly, as shown in the FIGs., the barbs 48 may be oriented in one direction facing toward the free end 50, 52 of the suture body 46.

The barbs 48 can be arranged in any suitable pattern along the suture body 46, for example, in a helical pattern as shown in FIG. 1. The number, configuration, spacing and surface area of the barbs 48 can vary depending upon one or more of the tissue in which the suture portion 34 is used, the specific surgical application, the composition of the suture body 46, and the geometry of the suture body 46. The proportions of the barbs 48 may remain relatively constant while the overall length and spacing of the barbs 48 are determined by the tissue being approximated to the matrix material 30. For example, if the suture portion 34 is intended to be used in muscle tissue, the barbs 48 can be made relatively short and more rigid to facilitate entry into this rather firm, fibrous tissue. If the suture portion 34 is intended for use in soft tissue, such as fat, the barbs 48 can be made longer and spaced farther apart to increase the holding ability in the soft tissue. In addition, for joining fat and relatively soft tissues, barbs 48 having a relatively large surface area are desired, whereas barbs 48 having a relatively smaller surface area are more suited for collagen-dense tissues. There are also situations where a combination of large and small surface area barbs 48 within the same structure will be beneficial, such as when the suture portion 34 is used in the repair of tissue with differing layered structures. Use of a combination of large and small surface area barbs 48 with the same suture portion 34, wherein barb 48 sizes are customized for each tissue layer, helps to ensure maximum anchoring properties. Particularly for when the body portion 32 comprises a scaffold, various barb geometries and arrays, as well as selected matrix construction, may be employed to help direct cell growth and aggregate, which should be better than with conventional tissue engineering scaffolds known in the art. Since cell growth includes initial adhesion as well as cell proliferation, certain cells may be induced to grow better with certain barb structures.

Examples of various barbed sutures suitable for use in the present invention include, but are not limited to, the barbed sutures described in U.S. Pat. No. 5,342,376, issued Aug. 30, 1994, and entitled “Inserting Device for a Barbed Tissue Connector”; U.S. Pat. No. 6,241,747, issued Jun. 5, 2001, and entitled “Barbed Bodily Tissue Connector”; U.S. Pat. No. 7,226,468, issued Jun. 5, 2007, and entitled “Barbed Bodily Tissue Connector”; U.S. Pat. No. 6,599,310, issued Jul. 29, 2003, and entitled “Suture Method”; U.S. Pat. No. 7,056,331, issued Jun. 6, 2006, and entitled “Suture Method”; U.S. Pat. No. 6,848,152, issued Feb. 1, 2005, and entitled “Method of Forming Barbs on a Suture and Apparatus for Performing Same”; U.S. Pat. No. 7,225,512, issued Jun. 5, 2007, and entitled “Method of Forming Barbs on a Suture and Apparatus for Performing Same”; and U.S. Pat. No. 5,931,855, issued Aug. 3, 1999, entitled “Surgical Methods Using One-Way Suture”. Also, suitable for use in the present invention are any of the barbed sutures, including embodiments comprising a surgical needle, as disclosed in U.S. Patent Application Pub No. 2004/0060409 A1, published April, 2004, and entitled “Barb Configurations for Barbed Sutures”; U.S. Patent Application Pub. No. 2004/0060410 A1, published Apr. 1, 2004, and entitled “Barbed Sutures”; and U.S. Patent Application Pub. No. 2004/0088003 A1, published May 16, 2004, and entitled “Barbed Suture in Combination with Surgical Needle”, may be employed. Each of the three publications contains an excellent discussion of various barb geometries and barb dispositions. Other medical devices and methods using babe sutures are disclosed in U.S. Pat. No. 6,773,450, issued Aug. 10, 2004, and entitled “Suture Anchor and Method”; U.S. Patent Application Pub. No. 2003/0074023 A1, published Apr. 17, 2003, and entitled “Suture Method”; and U.S. Patent Application Pub. No. 2005/0267531 A1, published Dec. 1, 2005, and entitled “Suture Methods and Devices”.

The barbs 48 may be formed on the surface of the suture body 46 according to any suitable method, including by cutting, by molding, by laser, and the like. One method is cutting with acute angular cuts directly into the suture body 46 with the cut portions pushed outwardly and separated from the body 46. The depth of the barbs 48 formed in the suture body 46 depends on the diameter of the suture material and the depth of cut. More particularly, embodiments of a suitable cutting device for cutting a plurality of axially spaced barbs 48 on the exterior of suture filaments are shown and described in U.S. Pat. No. 6,848,152, issued Feb. 1, 2005, and entitled “Method of Forming Barbs on a Suture and Apparatus for Performing Same”; U.S. Published Patent Application No. 2004/0237736, published Mar. 6, 2003, and entitled “Method of Forming Barbs on a Suture and Apparatus for Performing Same”; and U.S. Published Patent Application No. 2007/0065663 A1, published Mar. 22, 2007, and entitled “Apparatus for Forming Barbs on a Suture”. The cutting devices utilize a cutting bed, a cutting bed vise, a cutting template, and/or a blade assembly to perform the cutting. When operated, the cutting devices have the ability to produce a plurality of axially spaced barbs 48 in the same or random configuration and at different angles in relation to each other.

Various other suitable methods of cutting the barbs 48 have been proposed including the use of a laser. The barbs 48 could also be cut manually. However, manually cutting the barbs 48 is labor intensive, decreases consistency, and is not cost effective. The suture portion 34 could also be formed by injection molding, extrusion, stamping and the like.

As shown in the FIGS., the ends 50, 52 of the suture body 46 may be adapted for penetrating tissue, for instance, may comprise a point 54, 56 for penetrating tissue. In one embodiment, the portions of the ends 50, 52 of the suture body 46 adjacent the points 54, 56 may be formed of a material sufficiently stiff to enable the points 54, 56 to penetrate tissue in which the suture portion 34 is used when a substantially axial force is applied to the body 46. The ends 50, 52 of the suture portion 34 may be curved (FIGS. 1 and 2) or straight (FIGS. 3 and 4).

In another embodiment, one or both of the ends 50, 52 of the suture portion 34 may comprise a surgical needle 54, 56 for penetrating tissue. The needles are preferably constructed of stainless steel or other surgical-grade metal alloy, and may be curved (FIGS. 1 and 2) or straight (FIGS. 3 and 4). The length of the needles is selected to serve the type of tissue being repaired so that the needles can be completely removed leaving the suture body 46 in the desired position within the tissue. The needles may be attached to the suture body 46, as is known in the art, by means of adhesives, crimping, swaging, channel wrapping, heat shrinking, eyelet threading or the like. Also, as known in the art, a detachable connection may be employed such that the needles may be removed from the suture body 46 by a sharp tug or pull, or by cutting.

The suture portion 34 is attached to the body portion 32. Attachment may be achieved in a number of ways. By “attached”, “attachment”, and like terms, it is intended to include, but not to be limited to, unitary construction or individual construction. As best seen in FIGS. 1 and 2, the body portion 32 and the suture portion 34 may be of unitary construction, by which is meant that the body portion 32 and the suture portion 34 are formed in one unitary piece in the manufacturing process. In this embodiment, one or more such sutures could be woven or braided into the matrix material 30 during manufacture of the matrix material 30, typically with barbed portions 58, 60 of the suture or sutures being external to the body portion 32. Alternatively, the body portion 32 may be fabricated in one unitary piece such that one or more lengths of the suture body 46 extend outside the bulk of the body portion 32, and subsequently, the barbs 48 are selectively formed on the suture body 46, resulting in the barbed suture portion 34 with barbs 48 external to the body portion 32. Additionally, the body portion 32 and the suture portion 34 could be formed in one unitary piece by injection molding, extrusion, stamping, and the like.

The body portion 32 and the suture portion 34 may also be individually constructed and then attached to each other. For example, the suture portion 34 may be attached to the body portion 32 by inserting a proximal end (not shown) of each suture body 46 into an aperture 35 formed in the body portion 32, or the proximal end of the suture body 46 may itself create an aperture 35 upon insertion. The suture body 46 may then be secured in place by knotting the suture, or with a set screw, rivet, or the like. Where the material of the body portion 32 is metal or a metal alloy, attachment may be by swaging or crimping. Where the one or both of the materials of the body portion 32 and the suture portion 34 is polymeric, attachment may be by heat sealing.

As seen in FIGS. 1-4, each suture body 46 comprises a free distal end 50, 52 extending outside the bulk of the body portion 32. The barbs 48 along the length 58 of the suture body 46 between the body portion 32 and a first end 50 may be oriented in one direction facing toward the first end 50 of the suture body 46. The barbs 48 are oriented so as to allow movement of the length 58 of the suture body 46 through tissue in a direction of movement of the first end 50 and prevent movement of the length 58 of the suture body 46 relative to the tissue in the opposite direction. Similarly, the barbs 48 along a second length 60 of the suture body 46 face in direction toward the second end 52 of the suture body 46. The barbs 48 on the second length 60 of the suture body 46 are oriented so as to allow movement of the second length 60 of the suture body 46 through tissue in a direction of movement of the second end 52 and prevent movement of the length 58 of the suture body 46 relative to the tissue in the opposite direction. The number of barbs 48 on the first length 58 and second length 60 of the suture body 46, and the lengths of each of the first 58 and second lengths 60 of the suture body 46, can vary depending on the surgical application and needs.

As described above, the suture portion 34 may selectively comprise one or more sutures attached to the body portion 32, depending upon the particular surgical or the laboratory application. It is understood that more or fewer sutures may be used in the matrix material 30 according to the present invention, and one or more of the sutures may have no barbs.

A wide variety of suitable materials are available for the body portion 32 or the suture portion 34 of the matrix material 30. The particular material chosen depends on strength and flexibility requirements, as well as the end use application, for example, surgical mesh, mesh graft, hernia repair or scaffolding. Moreover, the body portion 32 and the suture portion 34 may be a same material or a different material. More particularly, the body portion 32 or the suture portion 34 may comprise nonabsorbable material, bioabsorbable material, or combinations thereof. Nonabsorbable material is particularly useful for the body portion 32 when it comprises hernia repair material, whereas bioabsorbable material, which is absorbed by the body over time, is particularly useful for the body portion 32 when it comprises scaffolding material. It is to be understood that a hernia repair material may be a transvaginal tape, which is used to hold the urethra, when the bladder has fallen and is out of place (i.e., herniated) and creates stress that results in urinary incontinence.

Generally, nonabsorbable material may be synthetic polymer, metal (i.e., titanium, tantalum, etc.), metal alloy (i.e., stainless steel), ceramic, natural material (i.e., silk, cotton, etc.), or combinations thereof. Typical nonabsorbable polymers may include, but are not limited to, polyethylene, polypropylene (commercially available as MARLEX®, registered trademark of Phillips Petroleum company, or commercially available as PROLENE®, registered trademark of Johnson & Johnson), polyamide (also known as nylon), polyester (such as polyethylene terephthalate, abbreviated here as PET, for instance, PET is commercially available as MYLAR®, registered trademark of du Pont de Nemours, or such as DACRON®, registered trademark of du Pont de Nemours, or such as MERSILENE®, registered trademark of Ethicon, Inc.), polytetrafluoroethylene (abbreviated here as PTFE, for instance, PTFE is commercially available as TEFLON®, registered trademark of du Pont de Nemours), expanded polytetrafluoroethylene (abbreviated here as ePTFE, for instance, ePTFE is commercially available as GOR-TEX®, registered trademark of Gore), polyether-ester (such as polybutester, which is the condensation polymerization of dimethyl terephthalate, polytetramethylene ether glycol, and 1,4-butanediol, and which is marketed by Davis & Geck and by U.S. Surgical, companies owned by Tyco, under the name NOVAFIL®, which is a trademark registered to American Cyanamid for surgical sutures), polyacrylic (commercially available as ORLON®, expired trademark of du Pont de Nemours), polyvinyl (commercially available as IVALON®, registered trademark of Unipoint Industries, Inc., or commercially available under the trade name VINYON-N), polyurethane, or combinations thereof. GOR-TEX® is a very suitable nonabsorbable polymer material for the body portion 32 when the matrix material 30 is to remain permanently in the body, such as when used as a hernia repair material.

Generally, absorbable material may be natural material, synthetic polymer, or combinations thereof. Depending on the particular absorbable material selected, the degradation time in the tissue ranges from about 1 month to about 24 months, or longer. Selection of the particular bioabsorbable material may be determined by the desired absorption or degradation time period, which depends upon the anticipated healing time for the subject of the procedure, or the anticipated time for the scaffolding to be in the laboratory prior to securing to a patient during a surgical procedure.

An example of natural absorbable material is collagen. Surgical gut comprises primarily collagen, and may be marketed as plain, chromic, and mild chromic. The chromic types are due to treatment with chromium salts to cross-link the collagen protein chains. Surgical gut is marketed as SURGIGUT®, registered trademark of United States Surgical Corporation.

Typical absorbable polymers include, but are not limited to, polydioxanone (which is a polyether-ester), polylactide, polylactic acid, copolymer of lactic acid and glyocolic acid, polyglaxanone, polyglactin, polycaprolactone, polytrimethylene carbonate, polymers having ester units, polymers having pyrrole units (for instance, polypyrrolic acid), polymers having thiophene units, polyphosphazene, or combinations thereof. It is noted that lactide is the cyclic dimer of lactic acid and exists as two optical isomers, D and L. Examples of polymers having ester units include, but are not limited to, polyglycolide (also called polyglycolic acid), polyorthoesters, copolymer of polyalkylene glycol and an aromatic polyester, or combinations thereof. Commercially available examples of absorbable polymers suitable for use in the matrix material 30 of the present invention are polyglycolic acid (sold as DEXON®, registered trademark of American Cyanamid), polyglactin (sold as VICRYL®, registered trademark of Johnson & Johnson), polydioxanone (sold as PDS II, by Ethicon, Inc.), copolymer of about 67% glycolide and about 33% trimethylene carbonate (sold as MAXON®, registered trademark of American Cyanamid), and copolymer of about 75% glycolide and about 25% caprolactone (sold as MONOCRYL®, registered trademark of Johnson & Johnson).

The approximate degradation times of various biodegradable synthetic polymers are reported by Middleton and Tipton, “Synthetic Biodegradable Polymers as Medical Devices”, Medical Plastics and Biomaterials Magazine (March, 1998). These times are summarized below in Table A. It is noted that each time listed is the time to complete mass loss, and the time will also depend partly on the geometry of the material.

TABLE A

| Biodegradable Polymer | Degradation Time (months) |
|---|---|
| PGA | 6 to 12 |
| (L)-PLA | >24 |
| (DL)-PLA | 12 to 16 |
| PCL | >24 |
| PDO | 6 to 12 |
| Copolymer of PGA-PTMC | 6 to 12 |
| 85%/15% (DL)-PLG | 5 to 6 |
| 75%/25% (DL)-PLG | 4 to 5 |
| 65%/35% (DL)-PLG | 3 to 4 |
| 50%/50% (DL)-PLG | 1 to 2 |

It is contemplated that when the body portion 32 comprises scaffolding material that is to be used in a laboratory, for instance as a chondrogenic growth-supporting matrix of bioabsorbable material, particularly for tissue engineering, then the body portion 32 should be made of a bioabsorable material that dissolves relatively faster, and the suture portion 34 should be made of some other bioabsorbable material that dissolves relatively slower, or be made of nonabsorbable material. That way when, in the laboratory, tissue is growing for a period of time on the body portion 32 of the matrix material 30, the suture portion 34 should better retain its integrity for the later surgical procedure to place the matrix material 30 with the tissue into the patient.

It is additionally contemplated that there may be instances when it is desirable for the suture portion 34 to degrade relatively faster than the body portion 32. For example, in some types of surgery, a scaffold could be placed in a patient and then the sutures would degrade rapidly, leaving intact for a selected time period the scaffold with whatever biological factor that that scaffold is delivering to the patient. It is also contemplated that there may be instances when it is desirable for the suture portion 34 to degrade in essentially the same time as the body portion 32.

Additionally, one or more biological factors may be incorporated into the body portion 32 or into the suture portion 34 or into both as a part of the matrix material 30. Various biological factors are known in the art of surgery, and it is not intended to be limited to any particular factor. For instance, one or more germicides may be factors that can be incorporated into the body portion 32 or the suture portion 34 of the matrix material 30 to provide long lasting germicidal properties. Also, the body portion 32 or the suture portion 34 can contain various repair factors, including, but not limited to, growth factors, fibroblast growth factors, transforming growth factor-beta, insulin, insulin-like growth factor 1 & 2 (IGF), platelet-derived growth factor, or combinations thereof, as is well known in the medical arts.

In use, the matrix material 30 according to the present invention is adapted such that the suture portion 34 fixedly engages tissue in order to secure the body portion 32 relative to the tissue. In a surgical procedure, the matrix material 30 is inserted into the body of a patient. The matrix material 30 is brought into position in proximity to a selected tissue site, for example, a hernia site. Once the body portion 32 is properly positioned, the sutures of the suture portion 34 extending from the body portion 32 are used for surgical suturing thereby securing the matrix material 30 to the surrounding tissue. More specifically, the point 54 at one end 50 of a suture body 46 is inserted into the tissue such that the point 54 pierces the tissue and the barbs 48 on the portion 58 of the suture body 46 corresponding to the one end 50 yield toward the body 46 to facilitate movement of the suture body as it is drawn through the tissue in the direction of insertion. Similarly, another point 56 at an end 52 of a suture body 46 is also inserted into the tissue and advanced through the tissue in like manner. The tissue is then advanced along the lengths 58, 60 of the suture bodies 46 within the tissue to close the gap between the tissue and the body portion 32. The leading ends 50, 52 of the suture body 46 protruding from the tissue are then cut and discarded. The barbs 48 on each suture body 46 engage the surrounding tissue and maintain the body portion 32 in position adjacent to the tissue during healing. It is noted that in the FIGS., barbs 48 are oriented toward end 50, but for certain applications, it may be desired that the barbs 48 are oriented away from end 50, or oriented in various directions, and in that event, the barbed suture may be inserted into the tissue with an insertion device, as described in the above-noted U.S. Pat. No. 5,342,376.

In embodiments wherein the body portion 32 comprises a scaffold and the scaffold is being used for cell growth and maturation in a laboratory, the barbs 48 may be masked. The masking is subsequently removed for implantation of the tissue engineered resultant into a patient. Moreover, the external barbs 48 may be unoptimized, so that cell growth is not vitiated. As is known, cells need adjacent neighboring cells in order to fuse. Additionally, it is contemplated that the body portion 32 may be completely or partially fabricated from barbed sutures so that as a result, the body portion 32 itself will internally contain barbs 48, as well as the suture portion 34 will contain barbs 48.

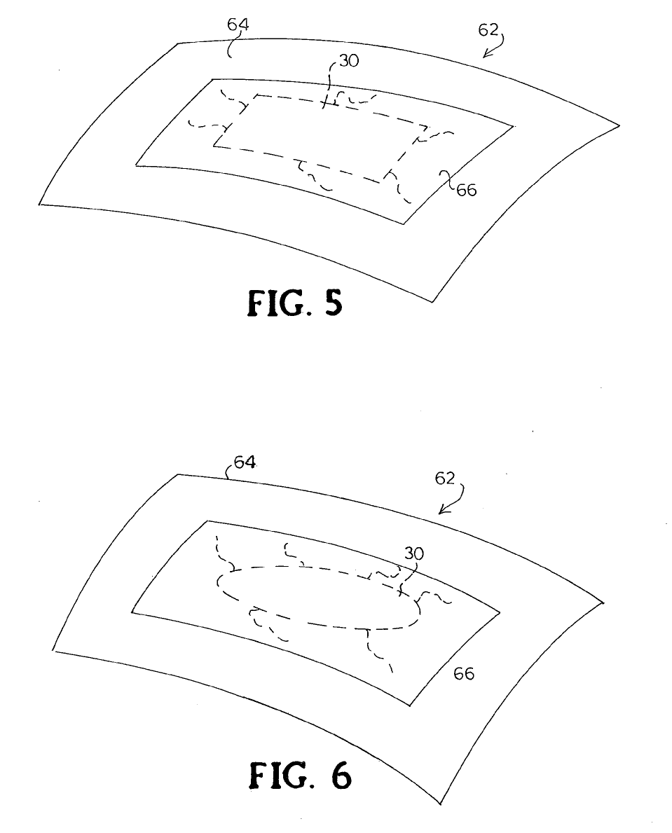

Additionally, the matrix material 30 according to the present invention may be disposed within a container. For instance, FIG. 5 schematically illustrates a package 62 having the matrix material 30 of FIG. 1 disposed within a container 64 having a sterile inside. FIG. 6 schematically illustrates a package 62 having the matrix material 30 of FIG. 2 disposed within a container 64 having a sterile inside. The matrix material 30 of FIG. 3 or FIG. 4 may be similarly disposed within a container (not shown). Such packaging of the matrix material 30 is preferred for ease of shipping to the end users and for preventing contamination during shipping to the end users, namely, various medical personnel, such as laboratory technicians, physicians, hospitals, etc. When the matrix material 30 is needed, medical personnel can remove the matrix material 30 from its sterile packaging 62. The matrix material 30 may be any of those described above or any obvious modifications of those described above, and should be sterile. The container 64 is sterile on the inside, and in one embodiment may be a clear see-through blister packaging 66. Other packaging materials may be used, such as paper or metal foil, and the present invention is not intended to be limited to any particular kind of packaging materials. As known in the art, packages 62 may be separate, or may be joined by perforations (not shown), the latter particularly being common with blister packaging. Such packaging lends itself well to a one-time use of the matrix material 30, after which the packaging may be thrown away. Methods for packaging items for a medical use, where the item is sterilized and sealed inside a container with a sterile inside environment, for instance using ethylene oxide for the sterilization, are well known to those of ordinary skill in the art.

Although the present invention has been shown and described in considerable detail with respect to only a few exemplary embodiments thereof, it should be understood by those skilled in the art that it is not intend to limit the invention to the embodiments since various modifications, omissions and additions may be made to the disclosed embodiments without materially departing from the novel teachings and advantages of the invention, particularly in light of the foregoing teachings. Accordingly, it is intend to cover all such modifications, omissions, additions and equivalents as may be included within the spirit and scope of the invention as defined by the following claims.

What is claimed is:

1. A matrix material for implanting in tissue in surgical applications, the matrix material comprising:
a body portion; and
at least one suture attached to the body portion so that the suture extends outwardly from the body portion, the at least one suture comprising an elongated suture body having a first distal end and a second proximal end, and a plurality of barbs projecting from the elongated suture body, each barb facing in a direction and being adapted for resisting movement, when in tissue, in an opposite direction from the direction in which the barb faces.

2. The matrix material as recited in claim 1, wherein the body portion comprises a material chosen from surgical mesh, mesh graft, sheeting, cloth, hernia repair material, scaffolding material, or combinations thereof.

3. The matrix material as recited in claim 1, wherein the barbs face the distal end of the at least one suture for permitting movement of the at least one suture through the tissue in a direction of movement of the distal end and preventing movement of the at least one suture relative to the tissue in a direction opposite the direction of movement of the distal end.

4. The matrix material as recited in claim 1, wherein the distal end of the at least one suture is adapted for penetrating tissue or the distal end of the at least one suture comprises a needle.

5. The matrix material as recited in claim 1, wherein the body portion and the at least one suture are a same material or a different material, the material being chosen from absorbable material, nonabsorbable material, or combinations thereof.

6. The matrix material as recited in claim 5, wherein the body portion and the at least one suture are chosen from polymer, metal, metal alloy, ceramic, natural material, or combinations thereof.

7. The matrix material as recited in claim 6, wherein the body portion and the at least one suture are chosen from tantalum, titanium, stainless steel, silk, cotton, collagen, polyethylene, polyester, polyether-ester, polyamide, polyvinyl, polypropylene, polytetrafluoroethylene, expanded polytetrafluoroethylene, polyacrylic, polyurethane, polydioxanone, polylactide, polylactic acid, polyglycolide, polycaprolactone, polytrimethylene carbonate, copolymer of lactic acid and glyocolic acid, polyglaxanone, polyglactin, polymers having pyrrole units, polymers having thiophene units, polyphosphazene, or combinations thereof.

8. The matrix material as recited in claim 1, wherein the body portion comprises absorbable scaffolding material having a certain degradation time, and the at least one suture comprises one or more of (i) nonabsorbable material or (ii) absorbable material having a degradation time that is slower than, faster than, or the same as the degradation time of the absorbable scaffolding material.

9. A surgical method for suturing to tissue a matrix material, the suturing method comprising the steps of:
(a) providing a matrix material including a body portion and including at least one suture attached to the body portion so that the suture extends outwardly from the body portion, the at least one suture comprising an elongated suture body having a first distal end and a second proximal end, and a plurality of barbs projecting from the elongated suture body, each barb facing in a direction and being adapted for resisting movement, when in tissue, in an opposite direction from the direction in which the barb faces;
(b) positioning the body portion in the tissue;
(c) inserting the distal end of the at least one suture into the tissue;
(d) pushing the distal end of the at least one suture through the tissue until the distal end of the suture extends out of the tissue at an exit point spaced from the point of insertion into the tissue; and
(e) gripping the distal end of the at least one suture and pulling the end out of the tissue for drawing the suture through the tissue while approximating the body portion adjacent a selected position in the tissue and leaving a length of the at least one suture in the tissue.

10. The suturing method as recited in claim 9, wherein:
the matrix material includes at least a second suture attached to the body portion so that the second suture extends outwardly from the body portion, the second suture comprising an elongated suture body having a first distal end adapted for penetrating tissue and a second proximal end and a plurality of barbs projecting from the elongated suture body, each barb facing in a direction and being adapted for resisting movement, when in tissue, in an opposite direction from the direction in which the barb; and
steps (c) through (e) are repeated with the second suture.

11. The suturing method as recited in claim 9, wherein the distal end of the at least one suture is adapted for penetrating tissue or the distal end of the at least one suture comprises a needle.

12. The suturing method as recited in claim 9, wherein the body portion and the at least one suture are a same material or a different material, said material being chosen from absorbable material, nonabsorbable material, or combinations thereof.

13. The suturing method as recited in claim 12, wherein the body portion and the at least one suture are chosen from polymer, metal, metal alloy, ceramic, natural material, or combinations thereof.

14. The suturing method as recited in claim 13, wherein the body portion and the at least one suture are chosen from tantalum, titanium, stainless steel, silk, cotton, collagen, polyethylene, polyester, polyether-ester, polyamide, polyvinyl, polypropylene, polytetrafluoroethylene, expanded polytetrafluoroethylene, polyacrylic, polyurethane, polydioxanone, polylactide, polylactic acid, polyglycolide, polycaprolactone, polytrimethylene carbonate, copolymer of lactic acid and glyocolic acid, polyglaxanone, polyglactin, polymers having pyrrole units, polymers having thiophene units, polyphosphazene, or combinations thereof.

15. The suturing method as recited in claim 9, wherein the body portion comprises a material chosen from surgical mesh, mesh graft, sheeting, cloth, hernia repair material, scaffolding material, or combinations thereof.

16. The suturing method as recited in claim 9, wherein the method comprises surgery chosen from one or more of microsurgery, laparoscopic surgery, endoscopic surgery, or arthroscopic surgery.

17. The suturing method as recited in claim 9, wherein the body portion comprises absorbable scaffolding material having a certain degradation time, and the at least one suture comprises one or more of (i) nonabsorbable material or (ii) absorbable material having a degradation time that is slower than, faster than, or the same as the degradation time of the absorbable scaffolding material.

18. A package comprising a container having a sterile inside environment and a sterile matrix material disposed inside the container, wherein the matrix material comprises:
a body portion; and
at least one suture attached to the body portion so that the suture extends outwardly from the body portion, the at least one suture comprising an elongated suture body having a first distal end and a second proximal end, and a plurality of barbs projecting from the elongated suture body, each barb facing in a direction and being adapted for resisting movement, when in tissue, in an opposite direction from the direction in which the barb faces.

19. The package as recited in claim 18, wherein the body portion and the at least one suture are a same material or a different material, said material being chosen from absorbable material, nonabsorbable material, or combinations thereof.

20. The package as recited in claim 19, wherein the body portion and the at least one suture are chosen from polymer, metal, metal alloy, ceramic, natural material, or combinations thereof.

21. The package as recited in claim 20, wherein the body portion and the at least one suture are chosen from tantalum, titanium, stainless steel, silk, cotton, collagen, polyethylene, polyester, polyether-ester, polyamide, polyvinyl, polytetrafluoroethylene, expanded polytetrafluoroethylene, polypropylene, polyacrylic, polyurethane, polydioxanone, polylactide, polylactic acid, polyglycolide, polycaprolactone, polytrimethylene carbonate, copolymer of lactic acid and glycolic acid, polyglaxanone, polyglactin, polymers having pyrrole units, polymers having thiophene units, polyphosphazene, or combinations thereof.

22. The package as recited in claim 18, wherein the body portion comprises a material chosen from surgical mesh, mesh graft, hernia repair material, scaffolding material, or combinations thereof.

23. The package as recited in claim 18, wherein the distal end of the at least one suture is adapted for penetrating tissue or the distal end of the at least one suture comprises a needle.

24. The package as recited in claim 18, wherein the body portion is chosen from absorbable scaffolding material having a certain degradation time, and the at least one suture is chosen from one or more of (i) nonabsorbable material, or (ii) absorbable material having a degradation time that is slower than, faster than, or the same as the degradation time of the absorbable scaffolding material.

25. The package as recited in claim 18, wherein the matrix material includes one or more biological factors may be incorporated into the body portion or the suture portion or a combination thereof.
